(19) United States
(12) Patent Application Publication
(10) Pub. No.: US 2026/0260743 A1
(43) Pub. Date: Sep. 3, 2026

(54) Systems and Methods for a Smart Walker to Predict Falls

(71) Applicants: Guhan Senthil, San Diego, CA (US); Declan Henckels, San Diego, CA (US)

(72) Inventors: Guhan Senthil, San Diego, CA (US); Declan Henckels, San Diego, CA (US)

(21) Appl. No.: 19/066,946

(22) Filed: Feb. 28, 2025

(51) Int. Cl. G16H 40/63

(52) U.S. Cl. CPC G16H 40/63

(57) ABSTRACT

A method, computer program product, and computer system for monitoring, by a computing device operatively connected to a walking assistance device, data associated with a walking pattern of a user. A change in the walking pattern of the user may be identified based upon, at least in part, the data. A likelihood of the user falling at a future time may be predicted based upon, at least in part, a value associated with the data being above at least one predetermined threshold. A warning may be provided to at least one of the user and a second user prior to the user falling based upon, at least in part, the value of the likelihood of the user falling at the future time being above the at least one predetermined threshold.

### BACKGROUND

Millions of people rely on walking assistance devices like walkers to maintain their mobility, including those with neurological conditions, individuals recovering from injuries, and the elderly. It is estimated that tens of thousands of falls occur annually in the U.S. alone, many of those falls being associated with a walker (or other walking assistance device). These falls cause injury and death, and are also costly in both in time and money. The risk of falling becomes incredibly stressful for the elderly, but also for caregivers. These concerns are heightened when walker users are left home alone, as it can take hours before a caregiver can learn about a fall and initiate help.

### SUMMARY

In one example implementation, a method, performed by one or more computing devices, may include but is not limited to monitoring, by a computing device operatively connected to a walking assistance device, data associated with a walking pattern of a user. A change in the walking pattern of the user may be identified based upon, at least in part, the data. A likelihood of the user falling at a future time may be predicted based upon, at least in part, a value associated with the data being above at least one predetermined threshold. A warning may be provided to at least one of the user and a second user prior to the user falling based upon, at least in part, the value of the likelihood of the user falling at the future time being above the at least one predetermined threshold.

One or more of the following example features may be included. The data may be received from a plurality of sensors operatively connected to the walking assistance device. At least a portion of the plurality of sensors may be located in a triangular arrangement on the walking assistance device. The plurality of sensors may include one or more distance sensors, one or more acceleration sensors, and one or more rotation sensors. Predicting the likelihood of the user falling at the future time may include conditioning the data of each sensor type received from of the plurality of sensors. Predicting the likelihood of the user falling at the future time may further include generating a respective score for each of the each sensor type. The at least one predetermined threshold may include a predetermined threshold for each of the each sensor type determined based upon, at least in part, conditioning of the data.

In another example implementation, a computing system may include one or more processors and one or more memories configured to perform operations that may include but are not limited to monitoring, by a computing device operatively connected to a walking assistance device, data associated with a walking pattern of a user. A change in the walking pattern of the user may be identified based upon, at least in part, the data. A likelihood of the user falling at a future time may be predicted based upon, at least in part, a value associated with the data being above at least one predetermined threshold. A warning may be provided to at least one of the user and a second user prior to the user falling based upon, at least in part, the value of the likelihood of the user falling at the future time being above the at least one predetermined threshold.

One or more of the following example features may be included. The data may be received from a plurality of sensors operatively connected to the walking assistance device. At least a portion of the plurality of sensors may be located in a triangular arrangement on the walking assistance device. The plurality of sensors may include one or more distance sensors, one or more acceleration sensors, and one or more rotation sensors. Predicting the likelihood of the user falling at the future time may include conditioning the data of each sensor type received from of the plurality of sensors. Predicting the likelihood of the user falling at the future time may further include generating a respective score for each of the each sensor type. The at least one predetermined threshold may include a predetermined threshold for each of the each sensor type determined based upon, at least in part, conditioning of the data.

In another example implementation, a computer program product may reside on a computer readable storage medium having a plurality of instructions stored thereon which, when executed across one or more processors, may cause at least a portion of the one or more processors to perform operations that may include but are not limited to monitoring, by a computing device operatively connected to a walking assistance device, data associated with a walking pattern of a user. A change in the walking pattern of the user may be identified based upon, at least in part, the data. A likelihood of the user falling at a future time may be predicted based upon, at least in part, a value associated with the data being above at least one predetermined threshold. A warning may be provided to at least one of the user and a second user prior to the user falling based upon, at least in part, the value of the likelihood of the user falling at the future time being above the at least one predetermined threshold.

One or more of the following example features may be included. The data may be received from a plurality of sensors operatively connected to the walking assistance device. At least a portion of the plurality of sensors may be located in a triangular arrangement on the walking assistance device. The plurality of sensors may include one or more distance sensors, one or more acceleration sensors, and one or more rotation sensors. Predicting the likelihood of the user falling at the future time may include conditioning the data of each sensor type received from of the plurality of sensors. Predicting the likelihood of the user falling at the future time may further include generating a respective score for each of the each sensor type. The at least one predetermined threshold may include a predetermined threshold for each of the each sensor type determined based upon, at least in part, conditioning of the data.

The details of one or more example implementations are set forth in the accompanying drawings and the description below. Other possible example features and/or possible example advantages will become apparent from the description, the drawings, and the claims. Some implementations may not have those possible example features and/or possible example advantages, and such possible example features and/or possible example advantages may not necessarily be required of some implementations.

### BRIEF DESCRIPTION OF DRAWINGS

FIG. 1 is an example diagrammatic view of a warning process implemented by an example distributed computing network according to one or more example implementations of the disclosure;

FIG. 2 is an example diagrammatic view of a client electronic device of FIG. 1 according to one or more example implementations of the disclosure;

FIG. 3 is an example flowchart of a warning process according to one or more example implementations of the disclosure;

FIG. 4 is an example diagrammatic view of a sample measurement table, walker and generic technique to measure sensor placement and angle according to one or more example implementations of the disclosure;

FIG. 5 is an example diagrammatic view of walkers with different sensors according to one or more example implementations of the disclosure;

FIG. 6 is an example diagrammatic view of a sample measurement table, walker and customized technique to measure sensor placement and angle according to one or more example implementations of the disclosure;

FIG. 7 is an example diagrammatic view of a fall prediction table according to one or more example implementations of the disclosure;

FIG. 8 is an example diagrammatic view of a screen image displayed by a warning process according to one or more example implementations of the disclosure;

FIG. 9 is an example diagrammatic view of an environment flowchart of a warning process according to one or more example implementations of the disclosure; and

FIG. 10 is an example diagrammatic view of hardware components of a monitoring devices used with a warning process according to one or more example implementations of the disclosure.

Like reference symbols in the various drawings may indicate like elements.

### DESCRIPTION

### System Overview:

In some implementations, the present disclosure may be embodied as a method, system, or computer program product. Accordingly, in some implementations, the present disclosure may take the form of an entirely hardware implementation, an entirely software implementation (including firmware, resident software, micro-code, etc.) or an implementation combining software and hardware aspects that may all generally be referred to herein as a “circuit,” “module” or “system.” Furthermore, in some implementations, the present disclosure may take the form of a computer program product on a computer-usable storage medium having computer-usable program code embodied in the medium.

Software may include artificial intelligence (AI) systems, which may include machine learning or other computational intelligence. For example, AI may include one or more models used for one or more problem domains. When presented with many data features, identification of a subset of features that are relevant to a problem domain may improve prediction accuracy, reduce storage space, and increase processing speed. This identification may be referred to as feature engineering. Feature engineering may be performed by users or may only be guided by users. In various implementations, a machine learning system may computationally identify relevant features, such as by performing singular value decomposition on the contributions of different features to outputs.

In some implementations, the various computing devices may include, integrate with, link to, exchange data with, be governed by, take inputs from, and/or provide outputs to one or more AI systems, which may include models, rule-based systems, expert systems, neural networks, deep learning systems, supervised learning systems, robotic process automation systems, natural language processing systems, intelligent agent systems, self-optimizing and self-organizing systems, and others. Except where context specifically indicates otherwise, references to AI, or to one or more examples of AI, should be understood to encompass one or more of these various alternative methods and systems; for example, without limitation, an AI system described for enabling any of a wide variety of functions, capabilities and solutions described herein (such as optimization, autonomous operation, prediction, control, orchestration, or the like) should be understood to be capable of implementation by operation on a model or rule set; by training on a training data set of human tag, labels, or the like; by training on a training data set of human interactions (e.g., human interactions with software interfaces or hardware systems); by training on a training data set of outcomes; by training on an AI-generated training data set (e.g., where a full training data set is generated by AI from a seed training data set); by supervised learning; by semi-supervised learning; by deep learning; or the like. For any given function or capability that is described herein, neural networks of various types may be used, including any of the types described herein, and in embodiments a hybrid set of neural networks may be selected such that within the set a neural network type that is more favorable for performing each element of a multi-function or multi-capability system or method is implemented. As one example among many, a deep learning, or black box, system may use a gated recurrent neural network for a function like language translation for an intelligent agent, where the underlying mechanisms of AI operation need not be understood as long as outcomes are favorably perceived by users, while a more transparent model or system and a simpler neural network may be used for a system for automated governance, where a greater understanding of how inputs are translated to outputs may be needed to comply with regulations or policies.

Examples of the models (e.g., AI-based models) include recurrent neural networks (RNNs) such as long short-term memory (LSTM), deep learning models such as transformers, decision trees, support-vector machines, genetic algorithms, Bayesian networks, and regression analysis. Examples of systems based on a transformer model include bidirectional encoder representations from transformers (BERT) and generative pre-trained transformers (GPT). Training a machine-learning model (or other type of AI-based learning models) may include supervised learning (for example, based on labelled input data), unsupervised learning, and reinforcement learning. In various embodiments, a machine-learning model may be pre-trained by their operator or by a third-party. Problem domains include nearly any situation where structured data can be collected, and includes natural language processing (NLP), including natural language understanding (NLU), computer vision (CV), classification, image recognition, etc. Some or all of the software may run in a virtual environment rather than directly on hardware. The virtual environment may include a hypervisor, emulator, sandbox, container engine, etc. The software may be built as a virtual machine, a container, etc. Virtualized resources may be controlled using, for example, a DOCKER container platform, a pivotal cloud foundry (PCF) platform, etc. Some or all of the software may be logically partitioned into microservices. Each microservice offers a reduced subset of functionality. In various embodiments, each microservice may be scaled independently depending on load, either by devoting more resources to the microservice or by instantiating more instances of the microservice. In various embodiments, functionality offered by one or more microservices may be combined with each other and/or with other software not adhering to a microservices model.

In some implementations, as noted above, AI-based learning models may include at least one of a transformer model, a convolutional neural network, a deep learning model trained on a set of outcomes of the value chain network entity, a supervised model, a semi-supervised model, an unsupervised model, or a reinforcement model, and the training data set for the AI-based learning models may include one or a set of objects or events that are labeled to classify the set of objects or events according to a classification taxonomy. Other examples of AI-based learning models (e.g., machine learning models) may include neural networks in general (e.g., deep neural networks, convolution neural networks, and many others), regression-based models, decision trees, hidden forests, Hidden Markov models, Bayesian models, and the like. In some implementations, the present disclosure may include combinations where an expert system uses one neural network for classifying an item and a different (or the same) neural network for predicting a state of the item.

In some implementations, any suitable computer usable or computer readable medium (or media) may be utilized. The computer readable medium may be a computer readable signal medium or a computer readable storage medium. The computer-usable, or computer-readable, storage medium (including a storage device associated with a computing device or client electronic device) may be, for example, but is not limited to, an electronic, magnetic, optical, electromagnetic, infrared, or semiconductor system, apparatus, device, or any suitable combination of the foregoing. More specific examples (a non-exhaustive list) of the computer-readable medium or storage device may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, solid state drives (SSDs), a digital versatile disk (DVD), a Blu-ray disc, and an Ultra HD Blu-ray disc, a static random access memory (SRAM), dynamic RAM (DRAM), synchronous DRAM (SDRAM), synchronous graphics RAM (SGRAM), and video RAM (VRAM), analog magnetic tape, digital magnetic tape, rotating hard disk drive (HDDs), a memory stick, a floppy disk, a mechanically encoded device such as punch-cards or raised structures in a groove having instructions recorded thereon, a media such as those supporting the internet or an intranet, or a magnetic storage device. Note that the computer-usable or computer-readable medium could even be a suitable medium upon which the program is stored, scanned, compiled, interpreted, or otherwise processed in a suitable manner, if necessary, and then stored in a computer memory. In the context of the present disclosure, a computer-usable or computer-readable, storage medium may be any tangible medium that can contain or store a program for use by or in connection with the instruction execution system, apparatus, or device.

Examples of storage implemented by the storage hardware include a distributed ledger, such as a permissioned or permissionless blockchain. Entities recording transactions, such as in a blockchain, may reach consensus using an algorithm such as proof-of-stake, proof-of-work, and proof-of-storage. Elements of the present disclosure may be represented by or encoded as non-fungible tokens (NFTs). Ownership rights related to the non-fungible tokens may be recorded in or referenced by a distributed ledger. Transactions initiated by or relevant to the present disclosure may use one or both of fiat currency and cryptocurrencies, examples of which include bitcoin and ether.

In some implementations, a computer readable signal medium may include a propagated data signal with computer readable program code embodied therein, for example, in baseband or as part of a carrier wave. In some implementations, such a propagated signal may take any of a variety of forms, including, but not limited to, electro-magnetic, optical, or any suitable combination thereof. In some implementations, the computer readable program code may be transmitted using any appropriate medium, including but not limited to the internet, wireline, optical fiber cable, RF, etc. In some implementations, a computer readable signal medium may be any computer readable medium that is not a computer readable storage medium and that can communicate, propagate, or transport a program for use by or in connection with an instruction execution system, apparatus, or device.

In some implementations, computer program code for carrying out operations of the present disclosure may be assembler instructions, instruction-set-architecture (ISA) instructions, machine instructions, machine dependent instructions, microcode, firmware instructions, state-setting data, state information that personalizes electronic circuitry and/or other structural components that are native to hardware (e.g., host processor, central processing unit/CPU, microcontroller, etc.) or either source code or object code written in any combination of one or more programming languages, including an object oriented programming language such as Java®, Smalltalk, C++ or the like. Java® and all Java-based trademarks and logos are trademarks or registered trademarks of Oracle and/or its affiliates. However, the computer program code for carrying out operations of the present disclosure may also be written in conventional procedural programming languages, such as the “C” programming language, PASCAL, or similar programming languages, as well as in scripting languages such as JavaScript, PERL, or Python. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a network, such as a cellular network, local area network (LAN), a wide area network (WAN), a body area network BAN), a personal area network (PAN), a metropolitan area network (MAN), etc., or the connection may be made to an external computer (for example, through the internet using an Internet Service Provider).

The networks may include one or more of point-to-point and mesh technologies. Data transmitted or received by the networking components may traverse the same or different networks. Networks may be connected to each other over a WAN or point-to-point leased lines using technologies such as Multiprotocol Label Switching (MPLS) and virtual private networks (VPNs), etc. In some implementations, electronic circuitry including, for example, programmable logic circuitry, an application specific integrated circuit (ASIC), gate arrays such as field-programmable gate arrays (FPGAs) or other hardware accelerators, micro-controller units (MCUs), or programmable logic arrays (PLAs), integrated circuits (ICs), digital circuit elements, analog circuit elements, combinational logic circuits, digital signal processors (DSPs), complex programmable logic devices (CPLDs), memory chips, network chips, systems on chip (SoCs), SSD/NAND controller ASICs, and the like, etc. may execute the computer readable program instructions/code by utilizing state information of the computer readable program instructions to personalize the electronic circuitry, in order to perform aspects of the present disclosure. Configurable or fixed-functionality logic may be implemented with complementary metal oxide semiconductor (CMOS) logic circuits, transistor-transistor logic (TTL) logic circuits, or other circuits. Multiple components of the hardware may be integrated, such as on a single die, in a single package, or on a single printed circuit board or logic board. For example, multiple components of the hardware may be implemented as a system-on-chip. A component, or a set of integrated components, may be referred to as a chip, chipset, chiplet, or chip stack. Examples of a system-on-chip include a radio frequency (RF) system-on-chip, an AI system-on-chip, a video processing system-on-chip, an organ-on-chip, a quantum algorithm system-on-chip, etc.

Examples of processing hardware may include, e.g., a central processing unit (CPU), a graphics processing unit (GPU), an accelerator (e.g., an AI accelerator), an approximate computing processor, a quantum computing processor, a parallel computing processor, a neural network processor, a signal processor, a digital processor, an analog processor, a data processor, an embedded processor, a microprocessor, and a co-processor. The co-processor may provide additional processing functions and/or optimizations, such as for speed or power consumption. Examples of a co-processor include a math co-processor, a graphics co-processor, a communication co-processor, a video co-processor, and an AI co-processor.

In some implementations, the AI accelerator may include suitable logic, circuitry, and/or interfaces to accelerate artificial intelligence applications, such as, e.g., artificial neural networks, machine vision and machine learning applications, including through parallel processing techniques. In one or more examples, the AI accelerator may include hardware logic or devices such as, e.g., a GPU or an FPGA. The AI accelerator may be used with any of the devices, components, features or methods described herein.

In some implementations, the flowchart and block diagrams in the figures illustrate the architecture, functionality, and operation of possible implementations of apparatus (systems), methods and computer program products according to various implementations of the present disclosure. Each block in the flowchart and/or block diagrams, and combinations of blocks in the flowchart and/or block diagrams, may represent a module, segment, or portion of code, which comprises one or more executable computer program instructions for implementing the specified logical function(s)/act(s). These computer program instructions may be provided to a processor of a general purpose computer, special purpose computer, or other programmable data processing apparatus to produce a machine, such that the computer program instructions, which may execute via the processor of the computer or other programmable data processing apparatus, create the ability to implement one or more of the functions/acts specified in the flowchart and/or block diagram block or blocks or combinations thereof. It should be noted that, in some implementations, the functions noted in the block(s) may occur out of the order noted in the figures (or combined or omitted). For example, two blocks shown in succession may, in fact, be executed substantially concurrently, or the blocks may sometimes be executed in the reverse order, depending upon the functionality involved. In addition, in some of the drawings, signal conductor lines may be represented with lines. Some may be different, to indicate more constituent signal paths, have a number label, to indicate a number of constituent signal paths, and/or have arrows at one or more ends, to indicate primary information flow direction(s). This, however, should not be construed in a limiting manner. Rather, such added detail may be used in connection with one or more implementations to facilitate ease of understanding. Any represented lines, whether or not having additional information, may actually comprise one or more signals/information that may travel in multiple directions and may be implemented with any suitable type of signal scheme, e.g., digital or analog lines implemented with differential pairs, optical fiber lines, and/or single-ended lines, etc.

In some implementations, these computer program instructions may also be stored in a computer-readable memory that can direct a computer or other programmable data processing apparatus to function in a particular manner, such that the instructions stored in the computer-readable memory produce an article of manufacture including instruction means which implement the function/act specified in the flowchart and/or block diagram block or blocks or combinations thereof.

In some implementations, the computer program instructions may also be loaded onto a computer or other programmable data processing apparatus to cause a series of operational steps to be performed (not necessarily in a particular order) on the computer or other programmable apparatus to produce a computer implemented process such that the instructions which execute on the computer or other programmable apparatus provide steps for implementing the functions/acts (not necessarily in a particular order) specified in the flowchart and/or block diagram block or blocks or combinations thereof.

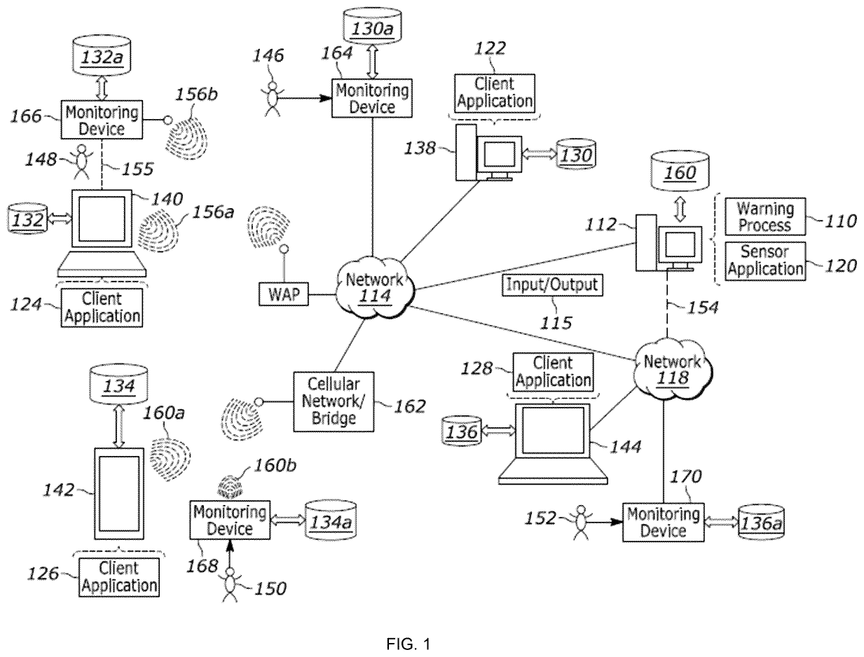

Referring now to the example implementation of FIG. 1, there is shown warning process 110 that may reside on and may be executed by a computer (e.g., computer 112), which may be connected to a network (e.g., network 114) (e.g., the internet or a local area network). Examples of computer 112 (and/or one or more of the client electronic devices noted below) may include, but are not limited to, a storage system (e.g., a Network Attached Storage (NAS) system, a Storage Area Network (SAN)), a personal computer(s), a laptop computer(s), mobile computing device(s), a server computer, a series of server computers, a mainframe computer(s), or a computing cloud(s). A SAN may include one or more of the client electronic devices, including a RAID device and a NAS system. In some implementations, each of the aforementioned may be generally described as a computing device. In certain implementations, a computing device may be a physical or virtual device. In many implementations, a computing device may be any device capable of performing operations, such as a dedicated processor, a portion of a processor, a virtual processor, a portion of a virtual processor, portion of a virtual device, or a virtual device.

In some implementations, a processor may be a physical processor or a virtual processor. In some implementations, a virtual processor may correspond to one or more parts of one or more physical processors. In some implementations, the instructions/logic may be distributed and executed across one or more processors, virtual or physical, to execute the instructions/logic. Computer 112 may execute an operating system, for example, but not limited to, Microsoft® Windows®; Mac® OS X®; Red Hat® Linux®, Windows® Mobile, Chrome OS, Blackberry OS, Fire OS, or a custom operating system. (Microsoft and Windows are registered trademarks of Microsoft Corporation in the United States, other countries or both; Mac and OS X are registered trademarks of Apple Inc. in the United States, other countries or both; Red Hat is a registered trademark of Red Hat Corporation in the United States, other countries or both; and Linux is a registered trademark of Linus Torvalds in the United States, other countries or both).

In some implementations, as will be discussed below in greater detail, a warning process, such as warning process 110 of FIG. 1, may monitor, by a computing device operatively connected to a walking assistance device, data associated with a walking pattern of a user. A change in the walking pattern of the user may be identified based upon, at least in part, the data. A likelihood of the user falling at a future time may be predicted based upon, at least in part, a value associated with the data being above at least one predetermined threshold. A warning may be provided to at least one of the user and a second user prior to the user falling based upon, at least in part, the value of the likelihood of the user falling at the future time being above the at least one predetermined threshold.

In some implementations, the instruction sets and subroutines of warning process 110, which may be stored on storage device, such as storage device 160, coupled to computer 112, may be executed by one or more processors and one or more memory architectures included within computer 112. In some implementations, storage device 160 may include but is not limited to: a hard disk drive; all forms of flash memory storage devices; a tape drive; an optical drive; a RAID array (or other array); a random access memory (RAM); a read-only memory (ROM); or combination thereof. In some implementations, storage device 160 may be organized as an extent, an extent pool, a RAID extent (e.g., an example 4D+1P R5, where the RAID extent may include, e.g., five storage device extents that may be allocated from, e.g., five different storage devices), a mapped RAID (e.g., a collection of RAID extents), or combination thereof.

In some implementations, network 114 may be connected to one or more secondary networks (e.g., network 118), examples of which may include but are not limited to: a local area network; a wide area network or other telecommunications network facility; or an intranet, for example. The phrase “telecommunications network facility,” as used herein, may refer to a facility configured to transmit, and/or receive transmissions to/from one or more mobile client electronic devices (e.g., cellphones, etc.) as well as many others.

In some implementations, computer 112 may include a data store, such as a database (e.g., relational database, object-oriented database, triplestore database, etc.), a data store, a data lake, a column store, and/or a data warehouse, and may be located within any suitable memory location, such as storage device 160 coupled to computer 112. In some implementations, data, metadata, information, etc. described throughout the present disclosure may be stored in the data store. In some implementations, computer 112 may utilize any known database management system such as, but not limited to, DB2, in order to provide multi-user access to one or more databases, such as the above noted relational database. In some implementations, the data store may also be a custom database, such as, for example, a flat file database or an XML database. In some implementations, any other form(s) of a data storage structure and/or organization may also be used. In some implementations, warning process 110 may be a component of the data store, a standalone application that interfaces with the above noted data store and/or an applet/application that is accessed via client applications 122, 124, 126, 128. In some implementations, the above noted data store may be, in whole or in part, distributed in a cloud computing topology. In this way, computer 112 and storage device 160 may refer to multiple devices, which may also be distributed throughout the network.

In some implementations, computer 112 may execute a monitoring application (e.g., monitoring application 120), examples of which may include, but are not limited to, a dashboard display system application designed to monitor and display sensor information to a user, such as a GUI or an aftermarket solution that can integrate with various sensors and provide real-time data, a digital dashboard application, a mobile application-based dashboard application, or other application that allows for monitoring, displaying, and/or providing alerts for sensor values, a data logging application to store historical data for analysis, a remote monitoring application to receive/send data from/to a cloud-based GUI for off-site access, a web conferencing application, a video conferencing application, a telephony application, a voice-over-IP application, a video-over-IP application, an Instant Messaging (IM)/“chat” application, a chatbot application, an interactive voice response (IVR) application, a short messaging service (SMS)/multimedia messaging service (MMS) application, or other application that allows for the monitoring, analysis, and response to sensor data. In some implementations, warning process 110 and/or monitoring application 120 may be accessed via one or more of client applications 122, 124, 126, 128. In some implementations, warning process 110 may be a standalone application, or may be an applet/application/script/extension that may interact with and/or be executed within monitoring application 120, a component of monitoring application 120, and/or one or more of client applications 122, 124, 126, 128. In some implementations, monitoring application 120 may be a standalone application, or may be an applet/application/script/extension that may interact with and/or be executed within warning process 110, a component of warning process 110, and/or one or more of client applications 122, 124, 126, 128. In some implementations, one or more of client applications 122, 124, 126, 128 may be a standalone application, or may be an applet/application/script/extension that may interact with and/or be executed within and/or be a component of warning process 110 and/or monitoring application 120. Examples of client applications 122, 124, 126, 128 may include, but are not limited to, e.g., a dashboard display system application designed to monitor and display sensor information to a user, such as a GUI or an aftermarket solution that can integrate with various sensors and provide real-time data, a digital dashboard application, a mobile application-based dashboard application, or other application that allows for monitoring, displaying, and/or providing alerts for sensor values, a data logging application to store historical data for analysis, a remote monitoring application to receive/send data from/to a cloud-based GUI for off-site access, a web conferencing application, a video conferencing application, a telephony application, a voice-over-IP application, a video-over-IP application, an Instant Messaging (IM)/“chat” application, a chatbot application, an interactive voice response (IVR) application, a short messaging service (SMS)/multimedia messaging service (MMS) application, or other application that allows for the monitoring, analysis, and response to sensor data, a chatbot application, a virtual assistant application, a sensor application, a standard and/or mobile web browser, an email application (e.g., an email client application), a textual and/or a graphical user interface, a customized web browser, a plugin, an Application Programming Interface (API), or a custom application. The instruction sets and subroutines of client applications 122, 124, 126, 128, which may be stored on storage devices 130, 132, 134, 136, 130a, 132a, 134a, 136a, coupled to client electronic devices 138, 140, 142, 144 and/or sensors 164, 166, 168, 170, may be executed by one or more processors and one or more memory architectures incorporated into client electronic devices 138, 140, 142, 144 and/or monitoring devices 164, 166, 168, 170.

In some implementations, one or more of storage devices 130, 132, 134, 136, 130a, 132a, 134a, 136a, may include but are not limited to: hard disk drives; flash drives, tape drives; optical drives; RAID arrays; random access memories (RAM); and read-only memories (ROM). Examples of client electronic devices 138, 140, 142, 144 (and/or computer 112) may include, but are not limited to, a personal computer (e.g., client electronic device 138), a laptop computer (e.g., client electronic device 140), a smart/data-enabled, cellular phone (e.g., client electronic device 142), a notebook computer (e.g., client electronic device 144), a tablet, a server, a television, a smart television, a smart speaker, an Internet of Things (IoT) device, a media (e.g., audio/video, photo, etc.) capturing and/or output device, an audio input and/or recording device (e.g., a handheld microphone, a lapel microphone, an embedded microphone/speaker (such as those embedded within eyeglasses, smart phones, tablet computers, smart televisions, a smart walking assistance device (e.g., a walker), wearables such as watches, headsets or glasses with a heads up display (HUD) capable of executing a virtual reality (VR) application, an extended reality (XR) application also known as mixed reality (MR), and/or an augmented reality (AR) application, health monitoring device, etc.), an infotainment device (e.g., such as those found in vehicles combining information and/or entertainment with optional screens and/or audio for such things as navigation, multimedia, connectivity, voice control, smartphone integration, touchscreen interface, internet and apps, rear-seat entertainment, etc.), a dedicated network device, and combinations thereof. Additionally/alternatively, one or more of client electronic devices 138, 140, 142, 144 may include a sensor or monitoring device (e.g., monitoring devices 164, 166, 168, 170), examples of which include but are not limited to motion sensors (e.g., accelerometers, gyroscopes, magnetometers, etc.), vision-based sensors (e.g., cameras-RGB, LiDAR, Time-of-Flight or other 3D motion tracking cameras, infrared, etc.), wearable sensors, pressure and force sensors, acoustic and sound sensors (microphones to detect known sounds such associated with a fall, such as a thud or cry for help, or ultrasonic sensors to detect movement patterns and sudden heigh changes using sound waves, biosensors (e.g., hear rate monitors, electromyography sensors, galvanic skin response sensors to measure skin conductivity changes due to stress or impact, etc.). Client electronic devices 138, 140, 142, 144 may each execute an operating system, examples of which may include but are not limited to, Android™, Apple® iOS®, Mac® OS X®; Red Hat® Linux®, Windows® Mobile, Chrome OS, Blackberry OS, Fire OS, or a custom operating system.

In some implementations, one or more of client applications 122, 124, 126, 128 may be configured to effectuate some or all of the functionality of warning process 110 (and vice versa). Accordingly, in some implementations, warning process 110 may be a purely server-side application, a purely client-side application, or a hybrid server-side/client-side application that is cooperatively executed by one or more of client applications 122, 124, 126, 128 and/or warning process 110.

In some implementations, one or more of client applications 122, 124, 126, 128 may be configured to effectuate some or all of the functionality of monitoring application 120 (and vice versa). Accordingly, in some implementations, monitoring application 120 may be a purely server-side application, a purely client-side application, or a hybrid server-side/client-side application that is cooperatively executed by one or more of client applications 122, 124, 126, 128 and/or monitoring application 120. As one or more of client applications 122, 124, 126, 128, warning process 110, and monitoring application 120, taken singly or in any combination, may effectuate some or all of the same functionality, any description of effectuating such functionality via one or more of client applications 122, 124, 126, 128, warning process 110, monitoring application 120, or combination thereof, and any described interaction(s) between one or more of client applications 122, 124, 126, 128, warning process 110, monitoring application 120, or combination thereof to effectuate such functionality, should be taken as an example only and not to limit the scope of the disclosure.

In some implementations, one or more of users 146, 148, 150, 152 and/or one or more of monitoring devices 164, 166, 168, 170 may access computer 112 and warning process 110 (e.g., using one or more of client electronic devices 138, 140, 142, 144) directly through network 114 or through network 118. Further, computer 112 may be connected to network 114 through network 118, as illustrated with phantom link line 154. Warning process 110 may include one or more user interfaces, such as browsers and textual or graphical user interfaces, through which users 146, 148, 150, 152 may access warning process 110 and/or monitoring devices 164, 166, 168, 170.

In some implementations, one or more of the various client electronic devices and/or one or more of monitoring devices 164, 166, 168, 170 may be directly or indirectly coupled to network 114 (or network 118). For example, client electronic device 138 (e.g., personal computer) and monitoring device 164 are shown directly coupled to network 114 via a hardwired network connection. Further, notebook computer (e.g., client electronic device 144) and monitoring device 170 are shown directly coupled to network 118 via a hardwired network connection. Laptop computer (e.g., client electronic device 140) and monitoring device 166 are shown wirelessly coupled to network 114 via wireless communication channels 156a and 156b respectively established between laptop computer (e.g., client electronic device 140) and wireless access point (i.e., WAP 158) and between monitoring device 166 and WAP 158, which is shown directly coupled to network 114. WAP 158 may be, for example, an IEEE 802.11a, 802.11b, 802.11g, Wi-Fi®, RFID, and/or Bluetooth™ (including Bluetooth™ Low Energy) device that is capable of establishing wireless communication channel 156a between laptop computer (e.g., client electronic device 140) and WAP 158 and wireless communication channel 156b between monitoring device 166 and WAP 158 (e.g., Zigbee, Z-Wave, etc.). Additionally/alternatively, a monitoring device (e.g., monitoring device 166) may be directly (and/or wirelessly) coupled to a client electronic device (e.g., client electronic device 140) as illustrated with phantom link line 155. Thus, information may be communicated from a monitoring device (e.g., monitoring device 166) to a client electronic device (e.g., client electronic device 140), where the information may be communicated, e.g., to computer 112 via, e.g., a network (e.g., network 114). Smart phone (e.g., client electronic device 142) and monitoring device 168 are shown wirelessly coupled to network 114 via wireless communication channels 160a and 160b respectively established between smart phone (e.g., client electronic device 142) and cellular network/bridge 162 and monitoring device 168 and cellular network/bridge 162, which is shown by example directly coupled to network 114.

In some implementations, some or all of the IEEE 802.11x specifications may use Ethernet protocol and carrier sense multiple access with collision avoidance (i.e., CSMA/CA) for path sharing. The various 802.11x specifications may use phase-shift keying (i.e., PSK) modulation or complementary code keying (i.e., CCK) modulation, for example. Bluetooth™ (including Bluetooth™ Low Energy) is a telecommunications industry specification that allows, e.g., mobile phones, computers, smart phones, and other electronic devices (e.g., monitoring devices 164, 166, 168, 170) to be interconnected using a short-range wireless connection. Other forms of interconnection (e.g., Near Field Communication (NFC)) may also be used. In some implementations, computer 112 may be directed or controlled by an operator. Computer 112 may be hosted by one or more of assets owned by the operator, assets leased by the operator, and third-party assets. The assets may be referred to as a private, community, or hybrid cloud computing network or cloud computing environment. For example, computer 112 may be partially or fully hosted by a third-party offering software as a service (SaaS), platform as a service (PaaS), and/or infrastructure as a service (IaaS). Computer 112 may be implemented using agile development and operations (DevOps) principles. In some implementations, some or all of computer 112 may be implemented in a multiple-environment architecture. For example, the multiple environments may include one or more production environments, one or more integration environments, one or more development environments, etc.

In some implementations, various I/O requests (e.g., I/O request 115) may be sent from, e.g., client applications 122, 124, 126, 128 to, e.g., computer 112 (and vice versa). Examples of I/O request 115 may include but are not limited to, data write requests (e.g., a request that content be written to computer 112) and data read requests (e.g., a request that content be read from computer 112). Client electronic devices 138, 140, 142, 144 and/or computer 112 may also communicate audibly using an audio codec, which may receive spoken information from a user and convert it to usable digital information. An audio codec may likewise generate audible sound for a user, such as through a speaker, e.g., in a handset of a client electronic device. Such sound may include sound from voice telephone calls, may include recorded sound (e.g., voice messages, music files, etc.) and may also include sound generated by applications operating on the client electronic devices.

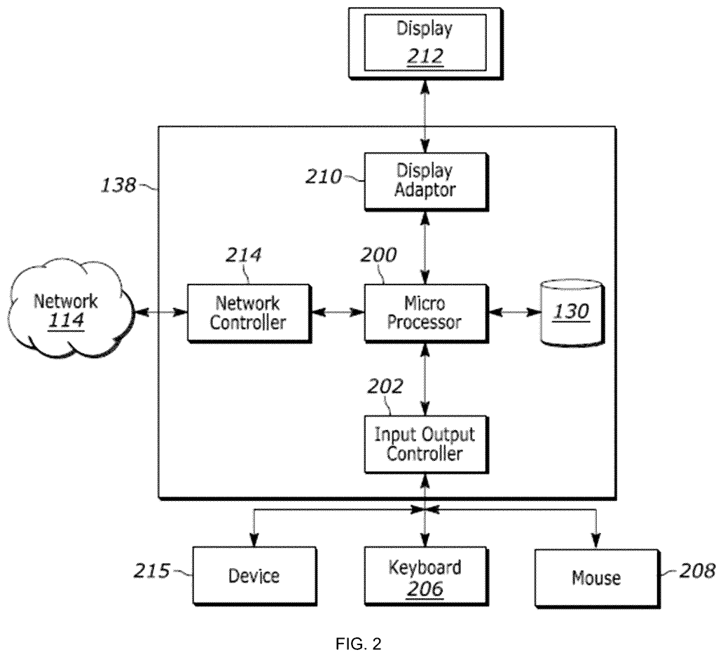

Referring also to the example implementation of FIG. 2, there is shown a diagrammatic view of client electronic device 138. While client electronic device 138 is shown in this figure, this is for example purposes only and is not intended to be a limitation of this disclosure, as other configurations are possible. Additionally, any computing device capable of executing, in whole or in part, warning process 110 may be substituted for client electronic device 138 (in whole or in part) within FIG. 2, examples of which may include but are not limited to computer 112 and/or one or more of client electronic devices 138, 140, 142, 144 and/or one or more of monitoring devices 164, 166, 168, 170.

In some implementations, client electronic device 138 may include a processor (e.g., microprocessor 200) configured to, e.g., process data and execute the above-noted code/instruction sets and subroutines. Microprocessor 200 may be coupled via a storage adaptor to the above-noted storage device(s) (e.g., storage device 130). An I/O controller (e.g., I/O controller 202) may be configured to couple microprocessor 200 with various devices (e.g., via wired or wireless connection), such as keyboard 206, pointing/selecting device (e.g., touchpad, touchscreen, mouse 208, etc.), scanner, custom device (e.g., device 215), USB ports, and printer ports. A display adaptor (e.g., display adaptor 210) may be configured to couple display 212 (e.g., touchscreen monitor(s), plasma, CRT, or LCD monitor(s), etc.) with microprocessor 200, while network controller/adaptor 214 (e.g., an Ethernet adaptor) may be configured to couple microprocessor 200 to network 114 (e.g., the Internet or a local area network).

As discussed above, millions of people rely on walking assistance devices like walkers to maintain their mobility, including those with neurological conditions, individuals recovering from injuries, and the elderly. It is estimated that tens of thousands of falls occur annually in the U.S. alone, many of those falls being associated with a walker (or other walking assistance device). These falls cause injury and death, and are also costly in both in time and money. The risk of falling becomes incredibly stressful for the elderly, but also for caregivers. These concerns are heightened when walker users are left home alone, as it can take hours before a caregiver can learn about a fall and initiate help. While some walkers may have sensors used to monitor the walker usage patterns to provide data on frequency, duration, and consistency of use, these sensors are thus attached to the wheels of the walker, so they do not monitor the user's gait or have the ability to predict that the user might fall in the near future.

Therefore, as will be discussed in greater detail below, the present disclosure helps to mitigate the risk of falling by predicting when a fall is imminent, with enough time to intervene by notifying both the user of the walker and a caregiver (e.g., EMT, elderly caregiver, family member, 911, etc.). In some implementations, the present disclosure may also detect falls and notify caregivers when they occur, expediting the process of receiving help. In such an embodiment, the present disclosure may be able to detect a fall when the walker has tipped over, but also in the situation where walker has not tipped over and remains upright.

### The Warning Process:

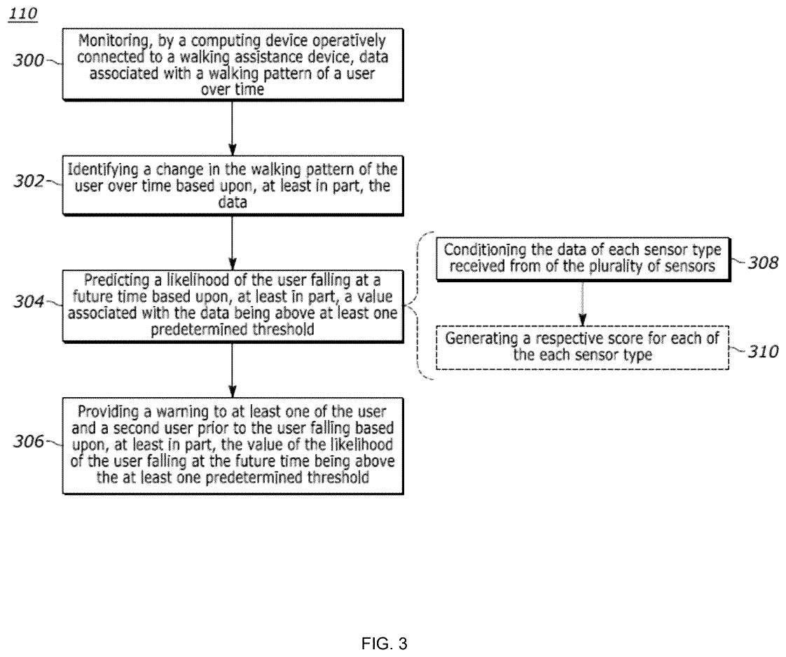

As discussed above and referring also at least to the example implementations of FIGS. 3-10, warning process 110 may monitor 300, by a computing device operatively connected to a walking assistance device, data associated with a walking pattern of a user. Warning process 110 may identify 302 a change in the walking pattern of the user based upon, at least in part, the data. Warning process 110 may predict 304 a likelihood of the user falling at a future time based upon, at least in part, a value associated with the data being above at least one predetermined threshold. Warning process 110 may provide 306 a warning to at least one of the user and a second user prior to the user falling based upon, at least in part, the value of the likelihood of the user falling at the future time being above the at least one predetermined threshold.

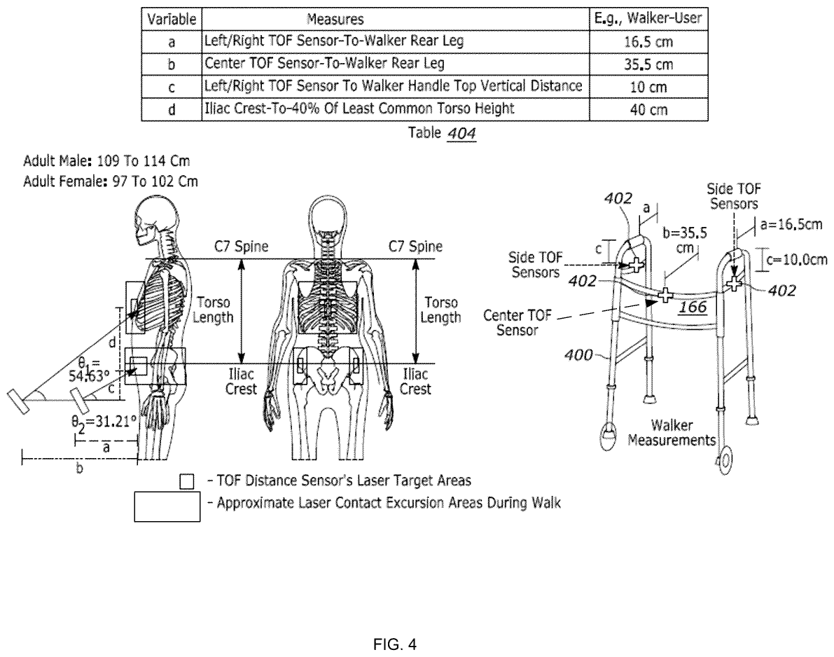

In some implementations, warning process 110 may monitor 300, by a computing device operatively connected to a walking assistance device, data associated with a walking pattern of a user. For instance, and referring at least to the example implementation of FIG. 4, assume for example purposes only that the walking assistance device is a walker (e.g., walker 400, shown as a standard foldable two-wheeled walker). In some implementations, warning process 110 may be operatively connected to walker 400 (e.g., via monitoring device 166) as an add-on attachment or as a built-in feature within the original design. As an add-on, monitoring devices 166 may be secured to walker 400 using various attachment methods, allowing for easy installation and removal. For example, monitoring devices 166 may be mounted with clamp-on attachments, which use adjustable clamps to fasten securely to the walker's frame, similar to accessory holders, hook-and-loop fastener straps or adhesive mounts can be used for lightweight sensor modules, offering flexibility in placement and easy repositioning, custom brackets or accessory rails can allow monitoring devices 166 to slide onto the walker's tubing, ensuring a secure fit.

For a more seamless integration, monitoring devices 166 can be built directly into the walker's design (e.g., using screws or any other known technique), offering a more aesthetically pleasing and functional approach. For instance, embedded frame sensors can be installed within the walker's tubing. In some implementations, walker 400 may include a built-in display and control panel placed on the handlebars to offer navigation assistance, step tracking, or other interactive features.

It will be appreciated after reading the present disclosure that other types of walking assistance devices may be used with warning process 110 without departing from the scope of the present disclosure. For instance, various types of walking assistance devices may include but are not limited to other walkers (e.g., standard walker, two-wheeled walker, four-wheeled rollator, hemi walker, knee walker), canes (e.g., standard cane, quad cane, folding cane, offset cane, smart cane), crutches (e.g., underarm crutches, forearm crutches, platform crutches), gait trainers and mobility aids (e.g., gait trainer, upright walker, exoskeleton), wheel-based mobility aids (e.g., manual wheelchair, powered wheelchair, mobility scooter). As such, the use of a standard foldable two-wheeled walker should be taken as example only and not to otherwise limit the scope of the present disclosure.

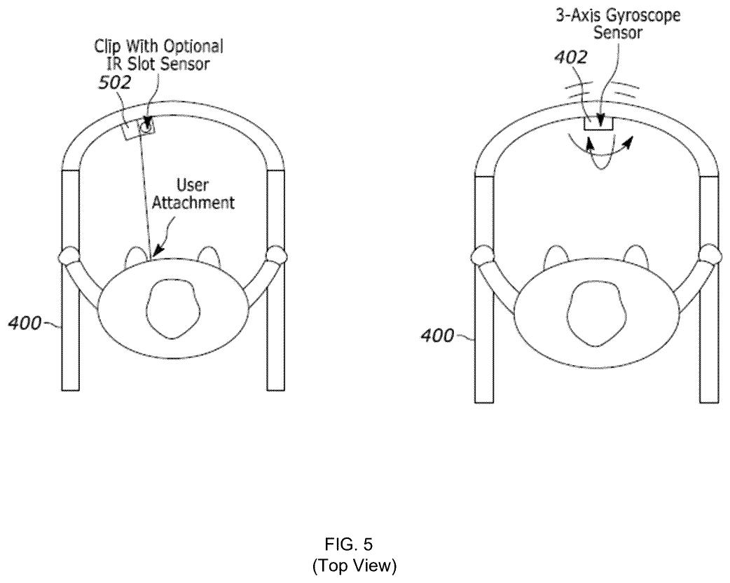

In some implementations, the data (e.g., IO request 115 from FIG. 1) may be received from a plurality of sensors operatively connected to the walking assistance device, and in some implementations, the plurality of sensors may include one or more distance sensors, one or more acceleration sensors, and one or more rotation sensors. For instance, the data used by warning process 110 to monitor the walking pattern of the user may be captured by and received from multiple sensors (e.g., sensor 402). Referring at least to the example implementations of FIG. 4 and FIG. 5, there is shown one or more Time-of-Flight (TOF) sensors to measure distance, one or more accelerometers used to detect changes in motion/tilt/velocity along one or more axis measuring how much the walker is speeding up or slowing in a particular direction, and one or more gyroscopes for real-time gait analysis by detecting rotational motion or changes in orientation around three axis, measuring how fast the walker is rotating or turning. It will be appreciated after reading the present disclosure that more or fewer sensors may be used without departing from the scope of the present disclosure. Each of these sensors (taken singly or in any combination) enable warning process 110 to learn the user's typical walk pattern, where the learned data is then used by warning process 110 to detect falls and predict the likelihood of a future fall, as will be discussed in greater detail below.

In some implementations, at least a portion of the plurality of sensors may be located in a triangular arrangement on the walking assistance device. For instance, as can be seen at least from the example implementations of FIG. 4, sensors 402 are located in a triangular arrangement (i.e., two side TOF sensors and a center TOF sensor). This specific configuration of sensors and sensor placement may be optimal for collecting the best data required for effective fall prediction at least because it ensures balanced, multi-directional data collection. A single sensor, whether placed in the center or on one side is possible, but it may miss asymmetrical movements or introduce bias, whereas a triangular setup captures movement from multiple angles, allowing for the detection of tilts, shifts, and irregular weight distributions that could indicate an impending fall. The side sensors monitor side-to-side swaying, which is an indicator of balance-related falls, while the center sensor detects forward or backward tilts, helping identify sudden leaning or loss of balance. Together, these sensors can triangulate the direction of instability, leading to more accurate fall predictions. In some implementations, the three TOF sensors may be used to create a 3D model of the user's torso movements, which may be used in the comparison of the learned profile and the real-time movement data when determining any change in the walking pattern of the user, described in more detail below. To help generate gait data for the user of the walker, warning process 100 may prioritize the torso, given its role in maintaining balance and aligning the center of gravity, making torso movement a more dependable indicator of gait changes than leg motion.

Additionally, a triangular arrangement improves weight distribution analysis by allowing warning process 110 to compare pressure differences between the left and right grips (when used), identifying irregular shifts in force that might signal a loss of balance. This setup also enhances machine learning and AI-based fall prediction algorithms of warning process 110 by providing multi-axis data, enabling warning process 110 to differentiate between normal movements and fall-risk behaviors with higher accuracy. Furthermore, having three sensors provides redundancy, ensuring that if one sensor fails, the other two can still supply enough data to detect potential falls. Cross-referencing sensor data also minimizes false positives, such as when a user shifts weight to sit down. By combining motion, force, and positional data, a triangular sensor placement to reliably measure torso movement significantly improves fall prediction reliability and overall user safety.

In some implementations, fall detection may also be provided. For instance, an IR sensor module with a magnetic latch (e.g., IR sensor module and magnetic latch 502 in FIG. 5) may be included, where disconnection of the latch confirms a fall. To make fall detection even more reliable, a clip using an IR-slot sensor may be used, similar to those used on treadmills. The clip may have a magnet on the walker side and is attached to the user, such that if the user falls, the magnet separates from the clip, triggering the IR sensor.

To guarantee accurate body movement capture, ~20 cm² area around the sensor's laser beam was used for reliable distance data when contacting any part of body. Two example and non-limiting methods for configuring the sensor's (e.g., TOF sensor's) orientation may be a generic and custom positioning. Caregivers can select the appropriate method based on their needs, making any necessary changes as desired.

The choice of sensors and their respective placement may help improve the accuracy of the data collected. For instance, and referring to the example implementation of FIG. 4, a table 404 of generic positioning of sensor(s) 400 is shown. It is recommended that walker 400 is adjusted so that the top of the handle is at the same height as the user's iliac crest, and the user's resting stance is recommended to be aligned with the walker's rear legs. Using the variables for table 404, as well as the formulas below, the angle placement of sensors 400 can be determined:

$$\begin{matrix}{θ}_{1} = {tan}^{- 1} ( \frac{( d + c )}{b} ) \times ( \frac{180 °}{π} ) \\ = {tan}^{- 1} ( \frac{40. + 10.}{35.5} ) \times ( \frac{180 °}{π} ) \\ = 54.63 °\end{matrix}$$

$$\begin{matrix}{θ}_{2} = {tan}^{- 1} ( \frac{c}{b} ) \times ( \frac{180 °}{π} ) \\ = {tan}^{- 1} ( \frac{10.}{16.5} ) \times ( \frac{180 °}{π} ) \\ = 31.21 °\end{matrix}$$

Based on the average torso length of adult males and females, the vertical distance between the laser focus point of the front and the iliac crest is set at 40 cm. This distance, representing approximately 40% of 100 cm (the least common average height of adult's torso), resulted in vertical angles of approximately 55° (θ₁=54.63°) for the front sensor and 30° (θ₂=31.210) for the side sensor(s). The side sensor's horizontal rotation was kept constant at 30°, a value determined experimentally for the walker used in earlier prototypes. In the example, the front sensor is positioned at a 90-degree angle to the walker frame, directed towards the user.

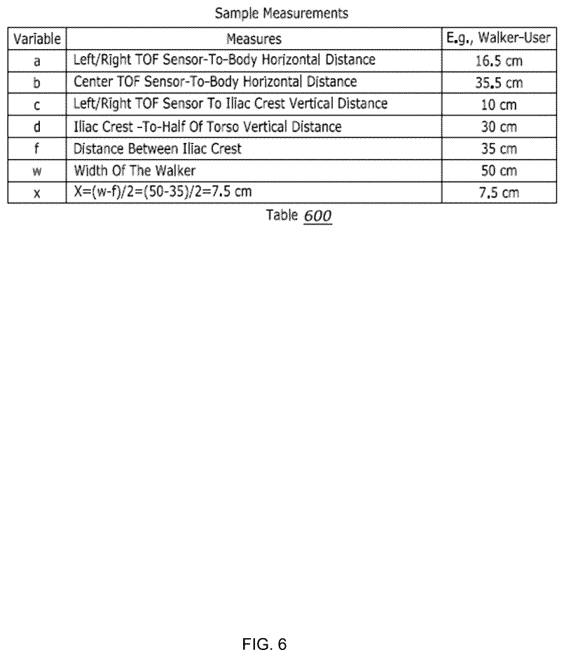

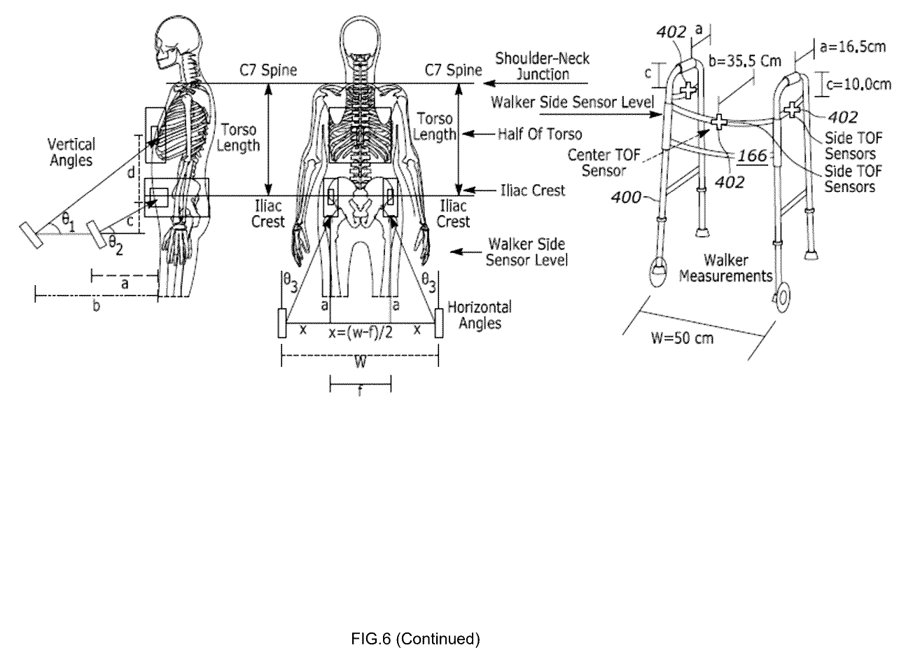

In some implementations, the generic sensor arrangement may not be suitable in all cases. To address this, a custom TOF sensor positioning methodology may also be selected. This method allows for individualized configuration based on the walker and the user's resting posture. By measuring distances to anatomical landmarks (e.g., half torso length and iliac crest), the optimal sensor angles for vertical and horizontal skew can be determined by warning process 110 as shown in the example FIG. 6, where a table 600 of custom positioning of sensor(s) 400 is shown. This approach does not have any assumptions about average torso length, and as such, can be adapted for any posture.

Using the variables for table 600, as well as the formulas below, the angle placement of sensors 400 can be determined:

$$\begin{matrix}{θ}_{1} = {tan}^{- 1} ( \frac{d + c}{b} ) \times ( \frac{180 °}{π} ) \\ = {tan}^{- 1} ( \frac{30. + 10.}{35.5} ) \times ( \frac{180 °}{π} ) \\ = 48.4 °\end{matrix}$$

$$\begin{matrix}{θ}_{2} = {tan}^{- 1} ( \frac{c}{a} ) \times ( \frac{180 °}{π} ) \\ = {tan}^{- 1} ( \frac{10.}{16.5} ) \times ( \frac{180 °}{π} ) \\ = 31.21 °\end{matrix}$$

$$\begin{matrix}{θ}_{3} = 90 ° - {tan}^{- 1} ( \frac{a}{x} ) \times ( \frac{180 °}{π} ) \\ = 90 ° - {tan}^{- 1} ( \frac{10.}{7.5} ) \times ( \frac{180 °}{π} ) \\ = 36.38 °\end{matrix}$$

For the example case depicted in FIG. 6:
Front sensor vertical angle ~50° (θ₁=48.4°).
Side sensor vertical angle ~30° (θ₂=31.21°).
Side sensors horizontal angle ~35° (θ₃=36.38°).
Front sensor is positioned at a 90-degree angle to the walker frame.

These innovative arrangements capture all relevant body movements while preventing hand interference during walker use.

In some implementations, warning process 110 may identify 302 a change in the walking pattern of the user based upon, at least in part, the data. For instance, each person has a unique walking pattern when using a walker, and warning process 110 learns that pattern by accumulating data from the above-noted sensors to create a user profile to improve detection accuracy. Anomalies in that pattern can be detected/identified before a fall (e.g., as far as 1-10 seconds or more before a fall occurs), allowing enough time to notify the user (and/or caregiver) to react and prevent a fall if unusual movements suggest a fall might happen soon, as will be discussed in greater detail below. The walking pattern is learned and tracked using sensor data and an algorithm of warning process 110 (e.g., AI algorithm).

In some implementations, predicting the likelihood of the user falling at the future time may include conditioning 308 the data of each sensor type received from of the plurality of sensors. For instance, in some implementations, the statistical AI learning model may be used by warning process 110 to generate user profiles from the conditioned sensor data. The distance, acceleration, and rotation sensors provide three sets of real-time data, each containing diverse values and inherent noise. To address this, warning process 110 may use a weighted moving average mechanism, where the weights are learned adaptively (e.g., the most recent data point is given the highest weight to ensure recency bias). For instance, raw (preconditioned) data from the distance sensors (right, left, and center), accelerometers (Acc_X, Acc_Y, Acc_Z), and gyroscopes (Rot_X, Rot_Y, Rot_Z) are all smoothed using the weighted moving average filter shown below:

$$Smooth = \frac{( {w}_{1} \cdot {X}_{l} + {w}_{2} \cdot {X}_{l - 1} + ⋯ + {w}_{N} \cdot {X}_{t - N + 1} )}{( {w}_{1} + {w}_{2} + ⋯ + {w}_{N} )}$$

Where:
w(x) represents the weights.
X(t) is the value at time,
N is the number of elements in the sample.

$$Distance Conditioned Data = Smooth ( Right Sensor ) + Smooth ( Left Sensor ) + Smooth ( Center Sensor )$$

$$Acceleration Conditioned Data = Smooth ( Acc\_X ) + Smooth ( Acc\_Y ) + Smooth ( Acc\_Z )$$

$$Rotation Conditioned Data = Smooth ( Rot\_X ) + Smooth ( Rot\_Y ) + Smooth ( Rot\_Z )$$

For each sensor, the three smoothed values are then combined to create a single, consolidated signal representing that sensor's conditioned aggregated data. This conditioned data helps with robust statistical analysis, and both the learning mode (to learn the user's normal walker pattern) and monitor mode (to determine a change in that walker pattern) may use this smoothing algorithm. It will be appreciated that other conditioning techniques may also be used without departing from the scope of the present disclosure. For instance, smoothed, exponential, and weighted moving averages, Kalman filtering, Savitzky-Golay filtering, and median filtering may also be used singly or in combination to balance real-time responsiveness, noise reduction, and event detection.

In learning mode, for each sensor modality (e.g., distance, acceleration, and rotation), the mean (μ) and standard deviation(s) may be determined by warning process 110 as follows:

The sample mean (μ) is calculated using the formula:

$$μ = \frac{{x}_{1} + {x}_{2} + … + {x}_{n}}{n}$$

where:
μ represents the sample mean.
x₁, x₂, . . . , xₙ are the individual data points.
n is the total number of data points in the sample.
   The population standard deviation (o) is determined using the formula:

$$σ = \sqrt{\frac{\sum {( {x}_{i} - μ )}^{2}}{N}}$$

where:
σ (sigma) is the population standard deviation.
xᵢ represents the individual data points in the population.
μ is the population mean.
N is the total number of data points in the population.

In some implementations, warning process 110 may predict 304 a likelihood of the user falling at a future time based upon, at least in part, a value associated with the data being above at least one predetermined threshold. For instance, predicting the likelihood of the user falling at the future time may include generating 310 a respective score (e.g., z-score) for each of the sensor types, where the at least one predetermined threshold may include a predetermined threshold for each of the sensor types determined based upon, at least in part, conditioning of the data. For example, in monitoring mode, warning process 110 may continuously or intermittently analyze incoming real-time conditioned data by calculating z-scores and comparing them to a predetermined threshold (i.e., from the learned user profile stored locally or remotely). After some data analysis, it can be determined that under normal conditions, each sensor has a unique range of z-scores, and different sensors exhibit varying degrees of z-score change. The distance z-score may be the most sensitive indicator, followed by rotation and acceleration z-scores, respectively.

The z-score (z) is calculated using the formula:

$$z = \frac{x - μ}{σ}$$

where:
z represents the z-score.
x is the raw score (the specific data point being analyzed).
μ is the population mean,
σ is the population standard deviation.

Thus, the z-score is a statistical measure that helps compare real-time sensor data (in monitoring mode) to previously recorded data from a user's normal walking pattern (in learning mode). During the learning mode, sensors collect data on the user's normal walking behavior, including distance, acceleration, and rotation sensor values. This data is used to calculate a mean (μ) and standard deviation (σ) for each sensor. Then, in monitoring mode, current real-time sensor readings are continuously compared to the learned mean using the z-score formula. If the z-score exceeds a predefined threshold, it indicates a significant deviation from normal movement, meaning that a fall may be imminent within the next few seconds. Each sensor has a different z-score threshold, and when enough sensors exceed their respective thresholds, warning process 110 will classify the event as either a future fall warning, or a current fall having occurred.

In some implementations, as discussed above, the z-score thresholds may be established by warning process 110 during the learning mode, where sensors collect baseline data on the user's normal walking patterns. During this phase, warning process 110 records multiple data points for each sensor, calculating the mean (μ) and standard deviation (c) over time. The threshold is then set based on statistical confidence intervals, often using a z-score threshold of 2 or 3, which corresponds to a 95% or 99.7% confidence level in a normal distribution. This means that any new sensor's value reading that deviates beyond 26 or 36 from the learned mean is considered abnormal and could indicate a future fall may occur in the near future.

Additionally, threshold values may be customized per user to account for individual differences in walking patterns, strength, and stability. Adaptive thresholding techniques can also be employed, where warning process 110 continuously updates its learned model over time to adjust for gradual changes in mobility. By fine-tuning these thresholds, warning process 110 minimizes false positives (incorrectly classifying normal movement as a potential indication of an imminent future fall) while ensuring true indications of an imminent future fall is accurately detected, ultimately improving fall prediction reliability.

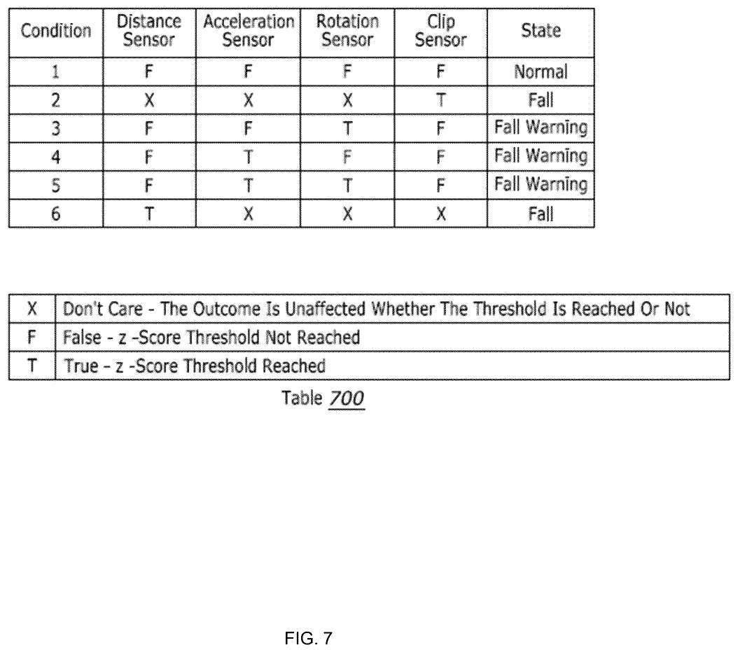

Referring at least to the example implementation of FIG. 7, an example fall detection/prediction truth table 700 is shown. The truth table details example logic for interpreting z-score data to trigger future fall warning (prediction) and fall detection. Table 700 defines different conditions based on sensor readings and classifies the user's state as Normal, Fall Warning, or Fall. In this particular example, it includes four types of sensors (e.g., distance sensor, acceleration sensor, rotation sensor, clip sensor). Each condition is evaluated based on whether a sensor's z-score threshold has been exceeded, helping to determine whether a fall is likely in the near future (e.g., within the next 10 seconds) or has already occurred.

In table 700, the symbols represent different interpretations of sensor readings. The “x” (Don't Care) symbol means the outcome does not depend on the value of that specific sensor, allowing flexibility in the decision-making process. “F” (False) indicates that the z-score threshold for that sensor was not reached, meaning the reading is within normal limits from the user profile data. “T” (True) signifies that the z-score threshold was reached, suggesting a significant deviation from the user's normal movement patterns in the user profile.

Each row in table 700 corresponds to a specific condition. Condition 1 represents normal walking behavior, where none of the sensors exceed their z-score thresholds (F, F, F, F) and the state is classified as NORMAL. Condition 2 classifies a FALL when the Clip Sensor detects a threshold breach (T) while the other sensors are marked as “x,” meaning their values are not necessary to confirm the fall. Condition 3 signals a FALL WARNING when only the rotation sensor exceeds its threshold (T) while the distance and acceleration sensors remain within normal limits. Conditions 4 and 5 also classify as FALL WARNING, as the acceleration sensor reaches its threshold (T), indicating sudden movement changes, while the other sensors remain normal or irrelevant. Condition 6 results in a FALL when the distance sensor alone reaches its threshold (T), suggesting a significant deviation in user position, even if the walker remains upright or even at a slight angle.

In some implementations, warning process 110 may provide 306 a warning to at least one of the user and a second user (e.g., a caregiver or someone else) prior to the user falling based upon, at least in part, the value of the likelihood of the user falling at the future time being above the at least one predetermined threshold. For instance, once warning process 110 predicts that the user is likely to fall in the near future, warning process 110 will send timely warnings to the walker user and/or a caregiver using intuitive and accessible alert methods. For the walker user, immediate alerts may allow them to react quickly and potentially prevent the fall. Haptic feedback, such as vibrations in the walker handles, smart phone, or a wearable device like a smartwatch, can provide a silent but noticeable warning, with different vibration patterns indicating varying levels of urgency. Audible alerts through a speaker on the walker or through a wirelessly connected hearing aid can deliver beeps, chimes, or verbal instructions (e.g., “Caution: Loss of Balance Detected”) to prompt corrective action. Visual indicators, such as LED warning lights on the walker or messages displayed on a built-in screen, can provide color-coded signals or text instructions. Additionally, a mobile device notification via a smartphone or smartwatch app can display a warning message with suggested actions, such as “Slow down” or “Grip walker handles firmly.”

For a caregiver (or family member or anyone else who is responsible for the walker user), real-time alerts can be beneficial, especially if the user has a history of instability. Text message alerts can provide instant notifications with location and sensor data (e.g., “Fall Warning: Unsteady movement detected for [User] at [Location]”). Email notifications, while not as beneficial in a time sensitive situation, can include more detailed reports, summarizing time, severity, and movement patterns over time for ongoing monitoring. Haptic alerts on the caregiver's smartwatch or smart ring can ensure they receive an alert even if their phone is not readily accessible. In critical situations, warning process 110 may auto-dial the caregiver's phone number or trigger an alert in a home monitoring system, such as smart speakers like Amazon Alexa or Google Home.

By combining multiple alert methods, warning process 110 helps ensure that both the walker user and caregiver receive timely and effective warnings. Haptic, visual, and audible alerts provide immediate feedback for the user, helping them adjust their movement, while text, email, and app notifications help ensure caregivers stay informed and can respond accordingly. These warnings can be customized based on urgency, user preference, and response protocols to enhance safety and reduce fall risks.

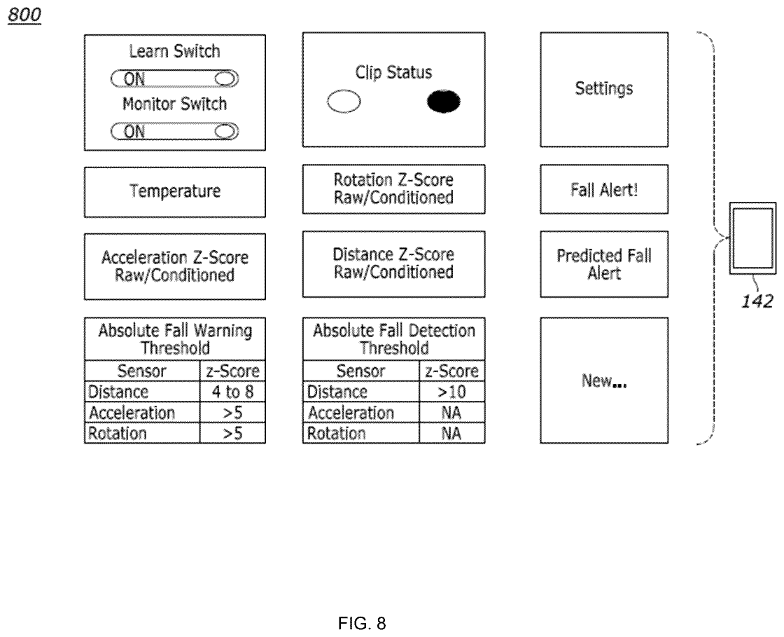

In some implementations, a caregiver app could also provide real-time updates, showing fall predictions, user location, and historical movement data to assist in proactive monitoring. An example of a dashboard 800 for the caregiver app is shown in the example implementation of FIG. 8. This caregiver dashboard/app view provides (e.g., via warning process 110) a real-time monitoring interface for tracking a walker user's movement and detecting predicted and actual falls. It displays critical sensor data, system status, and alerts, ensuring that caregivers can respond promptly to instability or fall events, while also monitoring past and current data. The Monitor Switch allows caregivers to enable or disable real-time fall monitoring, with warning process 110 actively processing sensor data when turned on. The Clip Status indicator shows whether the walker's clip sensor is engaged, providing insight into whether the user is actively using the walker. A Settings option gives access to configuration settings, such as adjusting fall detection thresholds, notification preferences, or sensor calibration.

In some implementations, several sections of the dashboard display key sensor readings. The Temperature section may provide environmental conditions relevant for health monitoring, while the Rotation Z-Score Raw/Conditioned section tracks rotational movement, helping detect abnormal shifts that may indicate a loss of balance. The Acceleration Z-Score Raw/Conditioned and Distance Z-Score Raw/Conditioned sections display real-time and processed sensor readings, monitoring sudden movements, deviations in walking patterns, and potential instability. A Predicted Fall Alert is triggered when sensor data suggests an imminent fall as discussed above, allowing caregivers to intervene before an accident occurs. If a fall is detected, the Fall Alert! section may provide a clear, high-priority notification requiring immediate attention.

The Absolute Fall Warning & Detection Thresholds tables outline the z-score thresholds used for classifying early fall prediction warnings versus actual falls. The Fall Warning Threshold indicates the early signs of instability, such as a distance z-score between 4 to 8 or a rotation z-score above 5. The Fall Detection Threshold signals a high probability of a fall, such as when the distance z-score exceeds 10. Lastly, a New . . . section may be a placeholder for additional features, such as new alerts, system updates, enhanced sensor visualization, etc.

In some implementations, warning process 110 may enable the detection of an actual fall, regardless of the movement and orientation of the walker itself, using a specific combination of sensors mounted to the walker. For example, as noted above, the IR sensor and magnetic latch may be used. However, by integrating distance, load, infrared, and optional wearable sensors, warning process 110 can accurately differentiate between normal walker movement and a real fall event. An ultrasonic or ToF distance sensor, mounted at hip level, can track the user's proximity to the walker. If the distance between the user and the walker suddenly increases beyond a normal range within a predetermined period of time, it may indicate a fall even if the walker remains upright. In some implementations, if the distance remains consistently low after this sudden increase, it suggests that the user is on the ground. Load sensors on the walker handles can further enhance detection by monitoring the user's grip pressure. A sudden loss of grip followed by no re-engagement may signal that the user has fallen and let go of the walker. Additionally, infrared or pressure mat sensors mounted on the walker base could detect whether the user is standing or has collapsed to the ground, further confirming a fall event.

In some implementations, the above-noted vision-based sensors may be used to provide evidence of a fall. For instance, vision-based sensors, such as cameras (e.g., RGB, depth, infrared, and thermal, etc.) combined with AI-driven computer vision models, may effectively detect if a person using a walker has fallen. Pose estimation models can track key body joints, such as the head, shoulders, hips, knees, torso, and feet, to analyze posture. Normal walker use generally involves an upright stance with symmetrical leg and arm movements, whereas a sudden collapse or an unusual body position, such as lying prone or supine on the ground, may indicate a fall. Angle-based heuristics can further refine detection, as a torso that is nearly parallel to the ground instead of vertical strongly suggests a fall.

Object detection models can recognize both the walker and the person using it. If the walker is detected without a person holding it or is overturned near a person lying on the ground, it raises a strong likelihood of a fall. Relative position tracking also helps, as a person being too far from their walker or in an unnatural position relative to it can be an indicator. Additionally, optical flow analysis can detect sudden, abrupt downward motion, which is often associated with falls. Motion recognition models, including Recurrent Neural Networks (RNNs) and Transformers, may be used to analyze movement sequences to differentiate between normal activities such as sitting or bending and an actual fall. Gait analysis models can also track movement patterns and detect sudden halts following irregular movement.

Depth sensors such as stereo cameras or LiDAR provide 3D spatial awareness, allowing systems to determine a person's position relative to the floor. If a person is lying flat instead of standing upright, it can signal a fall. TOF sensors may further enhance this by confirming whether a person's height has suddenly decreased drastically. In low-visibility conditions, thermal cameras can detect a person's body heat signature on the ground, compensating for the limitations of traditional RGB cameras.

For even greater accuracy, a wearable Inertial Measurement Unit (IMU), such as an accelerometer and gyroscope, attached to the user's wrist or belt, can detect sudden acceleration changes, free-fall motion, and impact forces. If the wearable sensor registers a fall while the walker's sensors confirm the user's absence, the likelihood of an actual fall is significantly increased.

Warning process 110 may integrate multiple detection steps to confirm a fall. First, the distance sensor detects an abnormal increase in user distance from the walker. Next, load sensors detect a loss of grip, suggesting that the user has let go. If additional sensors, such as pressure mats or infrared scanners, fail to detect the user standing, this further strengthens the classification of a fall. Finally, if the wearable IMU also detects a fall event, it serves as final confirmation that the user—not necessarily the walker—has fallen.

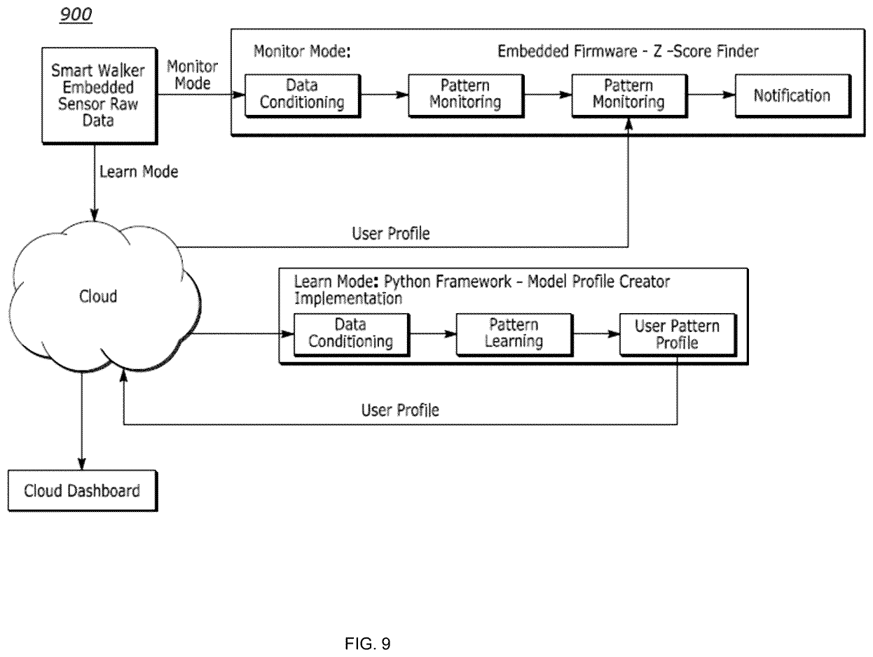

Referring at least to the example implementation of FIG. 9, an example and non-limiting diagrammatic view 900 of warning process 110 is shown. In some implementations, both embedded firmware and cloud-based learning models may be used to monitor user movement patterns and predict falls, as discussed above. As shown, warning process 110 operates in two primary modes: Learn Mode and Monitor Mode, both of which process sensor data from the walker to refine the user's movement profile and detect abnormalities.

In Learn Mode, raw sensor data from the smart walker may be sent to a cloud-based framework (e.g., python framework) that processes and conditions the data. Through pattern learning, warning process 110 analyzes the user's typical walking behavior and stores it as a User Pattern Profile. This profile serves as a reference model for detecting deviations that may indicate a predicted future fall or actual fall. The user profile created in this phase may then be stored and used for real-time monitoring.

In Monitor Mode, the embedded firmware on the walker may (e.g., via warning process 110) use the Z-Score Finder algorithm to continuously process incoming sensor data. This involves data conditioning, followed by pattern monitoring, where warning process 110 compares real-time movement against the previously learned user profile. If a deviation exceeds a certain threshold, indicating a potential fall risk, a notification is triggered to alert caregivers and/or the user. The user profile, developed in Learn Mode, ensures that fall prediction/detection is tailored to the individual's unique walking patterns rather than relying on generalized thresholds.

The network (e.g., cloud) may serve as a central hub, storing user profiles and facilitating access to the Cloud Dashboard, where caregivers or medical professionals can monitor the user's movement history, fall risk alerts, and overall mobility trends. This integration of embedded firmware and cloud-based learning ensures that the system not only reacts to predicted future and actual fall events but also adapts over time, improving the accuracy of fall prediction and reducing false alarms.

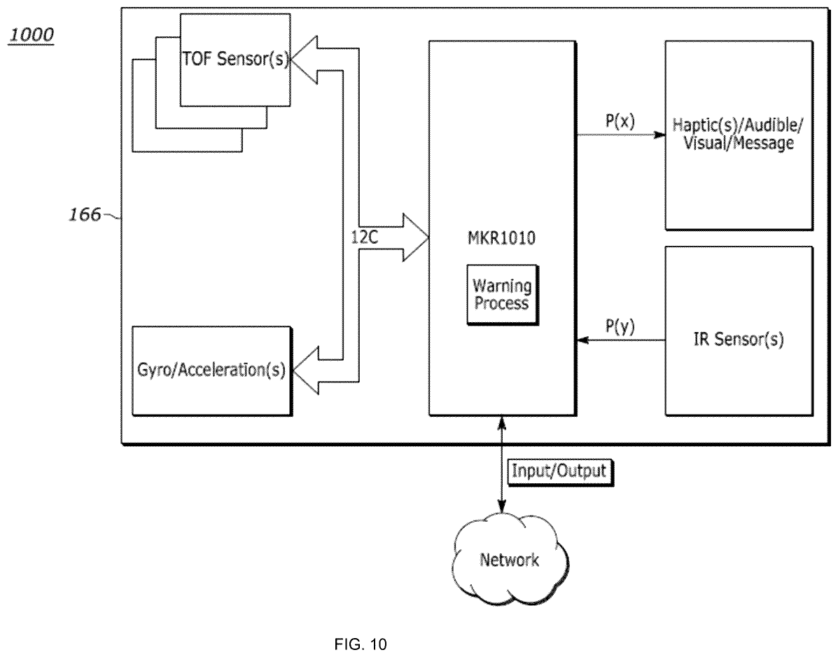

Referring to the example implementation of FIG. 10, an example diagrammatic view 1000 of the hardware components of monitoring devices 166 used with warning process 110 is shown. In the example, the sensor integration system is accomplished using a microcontroller (e.g., an Arduino MKR1010 microcontroller) to process data from multiple sensors and trigger output responses as discussed above. The system may use an Inter-Integrated Circuit (I2C) communication to gather data from various sensors, including ToF sensors and gyroscope/accelerometer modules, which track distance, motion, and orientation. The microcontroller acts as the central processing unit at least partially executing warning process 110, receiving real-time sensor data and analyzing it to determine appropriate responses.

As discussed above, once processed, warning process 110 sends output signals (e.g., haptic, audible, visual, messages, etc.) to feedback devices and infrared (IR) sensors via designated P (x) and P (y) output ports. The haptic feedback system can provide vibration alerts to the user, for warnings or notifications related to movement instability. Meanwhile, the IR sensors may serve functions such as proximity detection or environment monitoring. It will be appreciated after reading the present disclosure that various other or alternative components may also be used to carry out the teachings of warning process 110 without departing from the present disclosure. As such, the specific design of monitoring devices 166 from FIG. 10 should be taken as example only, and not to otherwise limit the scope of the present disclosure.

In some implementations, warning process 110 may be used as a valuable tool for physical therapy, rehabilitation, and walking aid evaluation, providing real-time data tracking, personalized feedback, and long-term progress analysis. For example, physical therapists and other medical providers can use warning process 110 to monitor a patient's mobility, balance, and gait patterns during rehabilitation. The device's embedded sensors, including ToF sensors, gyroscopes, accelerometers, and force sensors, can collect detailed movement data, helping therapists assess walking speed, step consistency, weight distribution, and stability over time. This data allows for objective progress tracking, enabling healthcare providers to compare a patient's mobility before and after therapy sessions.

Additionally, warning process 110 can provide real-time feedback to both the patient and therapist, alerting them to abnormal gait patterns, irregular weight shifts, or signs of fatigue. Haptic feedback, audio alerts, or visual indicators on a dashboard or mobile app can guide patients to make real-time adjustments to improve their walking technique. This is particularly beneficial for patients recovering from strokes, surgeries, or neurological conditions like Parkinson's disease. Warning process 110 may also be integrated with telemedicine platforms, allowing remote monitoring of rehabilitation progress, reducing the need for frequent in-person visits while ensuring continuous supervision by healthcare professionals.

Walking aid experts and mobility device providers can utilize warning process 110 to evaluate the effectiveness of walking aids, ensuring they are optimized for individual users. By analyzing sensor data, professionals can assess whether a particular walker properly supports the user's posture, weight distribution, and gait mechanics. If the data indicates uneven weight distribution, excessive reliance on one side, or difficulty maintaining balance, providers can make adjustments to the walker height, handle placement, or wheel configuration to improve comfort and support.

Moreover, warning process 110 can help determine whether a patient may need a different type of walking aid. If sensor readings consistently show high instability or frequent corrections, the patient might require a more supportive mobility device, such as a rollator, forearm walker, or even a transition to a wheelchair. Providers can also conduct comparative evaluations of different walkers, using objective data to recommend the best-suited walking aid for a particular user's needs.

The terminology used herein is for the purpose of describing particular implementations only and is not intended to be limiting of the disclosure. As used herein, the singular forms “a”, “an” and “the” are intended to include the plural forms as well, including any steps performed by a/the computer/processor, unless the context clearly indicates otherwise. As used herein, the phrase “at least one of A, B, and C” should be construed to mean a logical (A OR B OR C), using a non-exclusive logical OR, and should not be construed to mean “at least one of A, at least one of B, and at least one of C.” As another example, the language “at least one of A and B” (and the like) as well as “at least one of A or B” (and the like) should be interpreted as covering only A, only B, or both A and B, unless the context clearly indicates otherwise. It will be further understood that the terms “comprises” and/or “comprising,” when used in this specification, specify the presence of stated features, integers, steps (not necessarily in a particular order), operations, elements, and/or components, but do not preclude the presence or addition of one or more other features, integers, steps (not necessarily in a particular order), operations, elements, components, and/or groups thereof. Example sizes/models/values/ranges can have been given, although examples are not limited to the same.

The terms (and those similar to) “coupled,” “attached,” “connected,” “adjoining,” “transmitting,” “communicating,” “receiving,” “connected,” “engaged,” “adjacent,” “next to,” “on top of,” “above,” “below,” “abutting,” and “disposed,” used herein is to refer to any type of relationship, direct or indirect, between the components in question, and may apply to electrical, mechanical, fluid, optical, electromagnetic, electromechanical or other connections, including logical connections via intermediate components (e.g., device A may be coupled to device C via device B). Additionally, the terms “first,” “second,” etc. are used herein only to facilitate discussion, and carry no particular temporal or chronological significance unless otherwise indicated. The terms “cause” or “causing” means to make, force, compel, direct, command, instruct, and/or enable an event or action to occur or at least be in a state where such event or action is to occur, either in a direct or indirect manner. The term “set” does not necessarily exclude the empty set-in other words, in some circumstances a “set” may have zero elements. The term “non-empty set” may be used to indicate exclusion of the empty set—that is, a non-empty set must have one or more elements, but this term need not be specifically used. The term “subset” does not necessarily require a proper subset. In other words, a “subset” of a first set may be coextensive with (equal to) the first set. Further, the term “subset” does not necessarily exclude the empty set—in some circumstances a “subset” may have zero elements.

The corresponding structures, materials, acts, and equivalents (e.g., of all means or step plus function elements) that may be in the claims below are intended to include any structure, material, or act for performing the function in combination with other claimed elements as specifically claimed. While the disclosure describes structures corresponding to claimed elements, those elements do not necessarily invoke a means plus function interpretation unless they explicitly use the signifier “means for.” Unless otherwise indicated, recitations of ranges of values are merely intended to serve as a shorthand way of referring individually to each separate value falling within the range, and each separate value is hereby incorporated into the specification as if it were individually recited. While the drawings divide elements of the disclosure into different functional blocks or action blocks, these divisions are for illustration only. According to the principles of the present disclosure, functionality can be combined in other ways such that some or all functionality from multiple separately-depicted blocks can be implemented in a single functional block; similarly, functionality depicted in a single block may be separated into multiple blocks. Unless explicitly stated as mutually exclusive, features depicted in different drawings can be combined consistent with the principles of the present disclosure. Moreover, although this disclosure describes and depicts respective implementations herein as including particular components, elements, feature, functions, operations, or steps (and arrangements thereof), any of these implementations may include any combination, arrangement, or permutation of any of the components, elements, features, functions, operations, or steps described or depicted anywhere herein that a person having ordinary skill in the art would comprehend after reading the present disclosure. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative.

The description of the present disclosure has been presented for purposes of illustration and description but is not intended to be exhaustive or limited to the disclosure in the form disclosed. After reading the present disclosure, many modifications, variations, substitutions, and any combinations thereof will be apparent to those of ordinary skill in the art without departing from the scope and spirit of the disclosure. The implementation(s) were chosen and described in order to explain the principles of the disclosure and the practical application, and to enable others of ordinary skill in the art to understand the disclosure for various implementation(s) with various modifications and/or any combinations of implementation(s) as are suited to the particular use contemplated. The features of any dependent claim may be combined with the features of any of the independent claims or other dependent claims.

The implementations disclosed herein are only examples, and the scope of this disclosure is not limited to them. Implementations may include all, some, or none of the components, elements, features, functions, operations, or steps of the embodiments disclosed herein. The dependencies or references back in the attached claims are chosen for formal reasons only; however, any subject matter resulting from a deliberate reference back to any previous claims can be claimed as well, so that any combination of claims and the features thereof are disclosed and can be claimed regardless of the dependencies chosen in the attached claims. The subject-matter that can be claimed comprises not only the combinations of features as set out in the attached claims, but also any other combination of features in the claims, where each feature mentioned in the claims can be combined with any other feature or combination of other features in the claims. Furthermore, any of the implementations and features described or depicted herein can be claimed in a separate claim and/or in any combination with any implementation or feature described or depicted herein or with any of the features of the attached claims.

Having thus described the disclosure of the present application in detail and by reference to implementation(s) thereof, it will be apparent that modifications, variations, and any combinations of implementation(s) (including any modifications, variations, substitutions, and combinations thereof) are possible without departing from the scope of the disclosure defined in the appended claims.

What is claimed is:

1. A computer-implemented method comprising:
monitoring, by a computing device operatively connected to a walking assistance device, data associated with a walking pattern of a user over time;
identifying a change in the walking pattern of the user over time based upon, at least in part, the data;
predicting a likelihood of the user falling at a future time based upon, at least in part, a value associated with the data being above at least one predetermined threshold; and
providing a warning to at least one of the user and a second user prior to the user falling based upon, at least in part, the value of the likelihood of the user falling at the future time being above the at least one predetermined threshold.

2. The computer-implemented method of claim 1, wherein the data is received from a plurality of sensors operatively connected to the walking assistance device.

3. The computer-implemented method of claim 2, wherein at least a portion of the plurality of sensors are located in a triangular arrangement on the walking assistance device.

4. The computer-implemented method of claim 2, wherein the plurality of sensors includes one or more distance sensors, one or more acceleration sensors, and one or more rotation sensors.

5. The computer-implemented method of claim 4, wherein predicting the likelihood of the user falling at the future time comprises conditioning the data of each sensor type received from of the plurality of sensors.

6. The computer-implemented method of claim 5, wherein predicting the likelihood of the user falling at the future time further comprises generating a respective score for each of the each sensor type.

7. The computer-implemented method of claim 6, wherein the at least one predetermined threshold includes a predetermined threshold for each of the each sensor type determined based upon, at least in part, conditioning of the data.

8. A computer program product residing on a computer readable storage medium having a plurality of instructions stored thereon which, when executed across one or more processors, causes at least a portion of the one or more processors to perform operations comprising:
monitoring, by a computing device operatively connected to a walking assistance device, data associated with a walking pattern of a user;
identifying a change in the walking pattern of the user based upon, at least in part, the data;
predicting a likelihood of the user falling at a future time based upon, at least in part, a value associated with the data being above at least one predetermined threshold; and
providing a warning to at least one of the user and a second user prior to the user falling based upon, at least in part, the value of the likelihood of the user falling at the future time being above the at least one predetermined threshold.

9. The computer program product of claim 8, wherein the data is received from a plurality of sensors operatively connected to the walking assistance device.

10. The computer program product of claim 9, wherein at least a portion of the plurality of sensors are located in a triangular arrangement on the walking assistance device.

11. The computer program product of claim 9, wherein the plurality of sensors includes one or more distance sensors, one or more acceleration sensors, and one or more rotation sensors.

12. The computer program product of claim 11, wherein predicting the likelihood of the user falling at the future time comprises conditioning the data of each sensor type received from of the plurality of sensors.

13. The computer program product of claim 12, wherein predicting the likelihood of the user falling at the future time further comprises generating a respective score for each of the each sensor type.

14. The computer program product of claim 13, wherein the at least one predetermined threshold includes a predetermined threshold for each of the each sensor type determined based upon, at least in part, conditioning of the data.

15. A computing system including one or more processors and one or more memories configured to perform operations comprising:
monitoring, by a computing device operatively connected to a walking assistance device, data associated with a walking pattern of a user;
identifying a change in the walking pattern of the user based upon, at least in part, the data;
predicting a likelihood of the user falling at a future time based upon, at least in part, a value associated with the data being above at least one predetermined threshold; and
providing a warning to at least one of the user and a second user prior to the user falling based upon, at least in part, the value of the likelihood of the user falling at the future time being above the at least one predetermined threshold.

16. The computing system of claim 15, wherein the data is received from a plurality of sensors operatively connected to the walking assistance device, and wherein the plurality of sensors includes one or more distance sensors, one or more acceleration sensors, and one or more rotation sensors.

17. The computing system of claim 16, wherein at least a portion of the plurality of sensors are located in a triangular arrangement on the walking assistance device.

18. The computing system of claim 16, wherein predicting the likelihood of the user falling at the future time comprises conditioning the data of each sensor type received from of the plurality of sensors.

19. The computing system of claim 18, wherein predicting the likelihood of the user falling at the future time further comprises generating a respective score for each of the each sensor type.

20. The computing system of claim 19, wherein the at least one predetermined threshold includes a predetermined threshold for each of the each sensor type determined based upon, at least in part, conditioning of the data.
